(19) United States
(12) Patent Application Publication
(10) Pub. No.: US 2012/0277757 A1
(43) Pub. Date: Nov. 1, 2012

(54) MULTI-FUNCTION CANNULATED SURGICAL DEVICE

(75) Inventors: Amod P. Paranjpe, Augusta, MO (US); A. Jamie Riley, St. Louis, MO (US); Gary J. Schmidt, St. Louis, MO (US)

(73) Assignee: Curax, LLC, St. Louis, MO (US)

(21) Appl. No.: 13/446,885

(22) Filed: Apr. 13, 2012

Related U.S. Application Data
(60) Provisional application No. 61/474,964, filed on Apr. 13, 2011.
(60) Provisional application No. 61/514,702, filed on Aug. 3, 2011.

(51) Int. Cl. A61B 17/00

(52) U.S. Cl. 606/108

(57) ABSTRACT

A multi-function, cannulated, surgical device includes a cannulated member that has an inner cannula, having an opening therein to allow communication between an interior space of the inner cannula and an environment exterior to the cannulated member, and an outer cannula connected with the inner cannula; and wherein the inner and outer cannulas define a shared slot therebetween to allow communication between the interior space of the inner cannula and an interior space of the outer cannula.

### CROSS REFERENCES

This application claims priority to U.S. Provisional Patent Application No. 61/474,964, filed Apr. 13, 2011, and to U.S. Provisional Patent Application No. 61/514,702, filed Aug. 3, 2011.

### TECHNICAL FIELD OF THE INVENTION

The present invention relates generally to the field of surgical tools, for example, endoscopic, arthroscopic, and laparoscopic surgical tools and, more particularly, to a multi-function cannulated surgical device suitable for endoscopic, arthroscopic, and laparoscopic applications, among others.

### BACKGROUND OF THE INVENTION

In traditional surgical procedures involving endoscopy, arthroscopy or laparoscopy, at least two incisions are typically required. A first incision allows for the insertion of a scope while a second incision accommodates a surgical tool. The traditional methodology requires significant manual dexterity as the operating physician must generally operate both the scope and surgical tool simultaneously.

In addition, in many endoscopic, arthroscopic, and laparoscopic applications, the nature of the procedure and the treatment area results in very awkward positioning of the scope and surgical tool. For example, in the case of endoscopic plantar fasciotomy, the standard procedure requires insertion of the endoscope from one side of the patient's foot, while a cutting blade or other surgical tool is inserted from the opposite side of the foot. This orientation requires a difficult mental correction by the surgeon with respect to the direction of required movement of the surgical tool as the endoscopic is providing a view that is opposite to the orientation of the surgical tool.

The Agee Carpal Tunnel Release System, which was introduced in 1990, is the most recent pertinent advance within the field of endoscopic surgery. The Agee CTRS includes a video endoscope and a handpiece that holds an elongated, disposable blade assembly. This system does provide better alignment of the endoscopic view with the direction of operation of the blade assembly. However, the Agee CTRS is specifically designed for carpal tunnel release procedures and has limited applicability in other procedures. Further, the Agee device is expensive. Even more importantly, with the Agee device, the blade assembly may potentially come into contact with patient tissues unintentionally.

Therefore, it would be advantageous to provide a multi-function surgical device that allows for the introduction of multiple types of scopes and surgical tools from the same orientation without interfering with the physician's view of the treatment area and requiring a single incision.

### SUMMARY OF THE INVENTION

One aspect of the invention generally pertains to a multi-function cannulated surgical device that allows for the introduction of multiple surgical tools, including a scope, through a single tube structure.

Another aspect of the invention pertains to a multi-function cannulated surgical device that enables completion of certain procedures through a single incision.

Yet another aspect of the invention pertains to a multi-function cannulated surgical device that allows for the introduction of multiple surgical tools through a single incision while minimizing the possibility of unintended contact between those tools and patient tissues.

In accordance with the above aspects of the invention, there is provided a multi-function, cannulated, surgical device that includes a cannulated member that has an inner cannula, having an opening therein to allow communication between an interior space of the inner cannula and an environment exterior to the cannulated member, and an outer cannula connected with the inner cannula; and wherein the inner and outer cannulas define a shared slot therebetween to allow communication between the interior space of the inner cannula and an interior space of the outer cannula. In a preferred embodiment, the shared slot is formed by a passage having a width that is less than the diameter of the inner and outer cannulas and extending the entire length of the cannulas.

In another embodiment, the cannulated member may be combined with cartridge that is insertable within either the inner or outer cannula of the cannulated member and has a surgical tool connected to its distal end. The cartridge may also be connected with a surgical scope.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 is a top view of a multi-function, cannulated surgical device according to one exemplary embodiment of the present invention.

FIG. 2A is a side view of the device of FIG. 1.

FIG. 2B is enlarged detail view of a portion of a tab and slot arrangement utilized in the device of FIG. 1

FIG. 3 is a lateral cross-sectional view of the device of FIG. 1.

FIG. 4 is a perspective view from the proximal end of the device of FIG. 1.

FIG. 5 is a side view of a cartridge suitable for use with a multi-function cannulated surgical device such as is illustrated in FIGS. 1-4.

FIG. 6 is a top view of the cartridge of FIG. 5.

FIG. 7 is a top view of multi-function, cannulated surgical device according to another embodiment of the present invention.

FIG. 8 is an end view of the proximal end of the device of FIG. 7.

FIG. 9 is a side view of a cartridge suitable for use with a multi-function, cannulated surgical device according to another embodiment.

FIG. 10 is a top view of the cartridge of FIG. 9.

FIG. 11 is a top view of a multi-function, cannulated surgical device according to another embodiment.

FIG. 12 is a side view of a multi-function, cannulated surgical device with the cartridge in a first, lowered position relative to the cannulated member and resting in the outer cannula.

FIG. 13 is a side view of a multi-function, cannulated surgical device with the cartridge in a second, raised position relative to the cannulated member and resting in the inner cannula.

### DETAILED DESCRIPTION

In the following detailed description numerous specific details are set forth in order to provide a thorough understanding of the invention. However, it will be understood by those skilled in the art that the present invention may be practiced without these specific details. For example, the invention is not limited in scope to the particular type of industry application depicted in the figures. In other instances, well-known methods, procedures, and components have not been described in detail so as not to obscure the present invention.

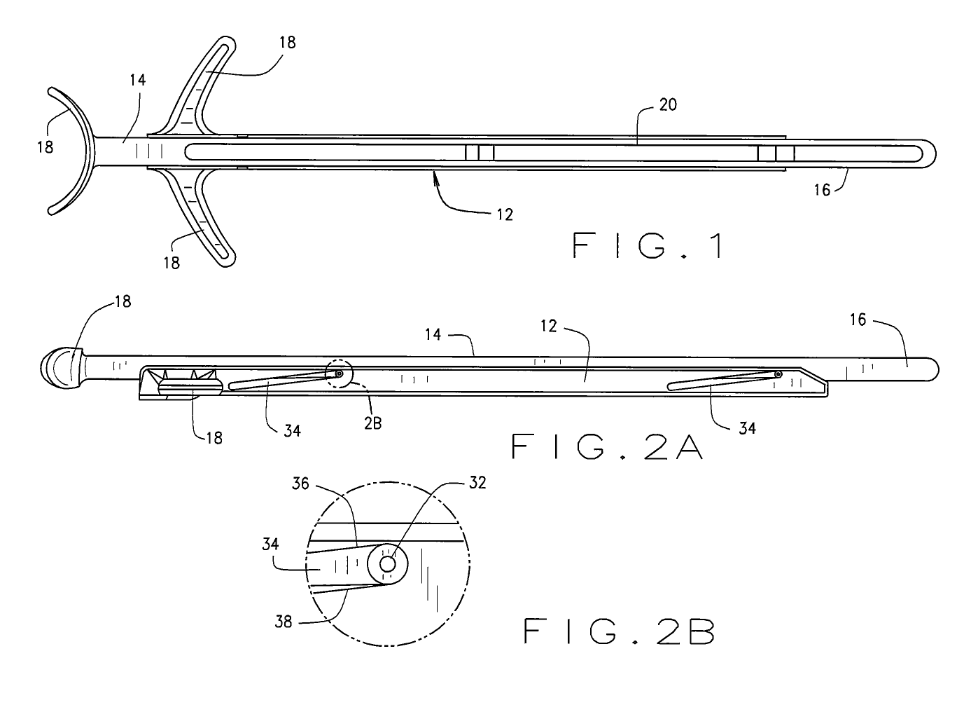

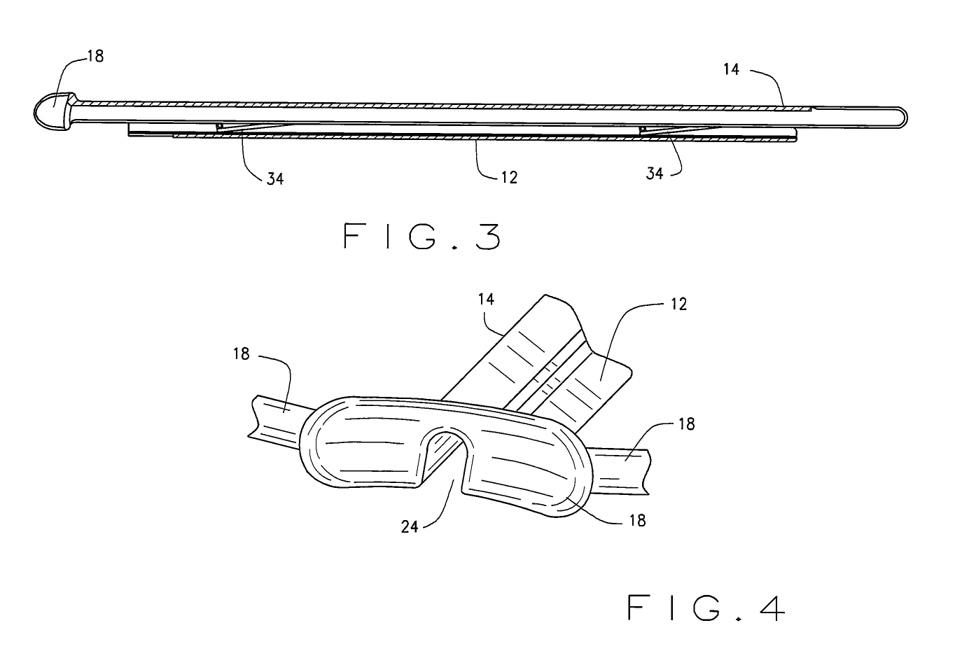

FIGS. 1-4 illustrate a multi-function, expandable, cannulated surgical device 10 according to an exemplary embodiment of the present invention. In pertinent part, the device 10 includes an outer cannula 12 that is in the form of an elongated partial tube with an open top. The device 10 further includes an inner cannula 14 in the form of an elongated tube with an open bottom. Both the inner 14 and outer 12 cannulas may be provided with multiple cross-sectional shapes, e.g., circular, oval, square, etc. However, the cross-sectional shapes of a given inner/outer cannula pair should match one another. Advantageously, the outer cannula 12 is provided with an inner dimension that allows the inner cannula 14 to fit and slide within the outer cannula 12.

The distal end of the inner cannula 14 is provided with a variety of different tips that may be selected based upon the procedure in which the device is being used. For example, in the illustrated embodiment, the inner cannula 14 is illustrated with a rounded tip 16 to ease insertion of the device through an incision. However, other options include a split, beveled or a spatula tip.

The proximal ends of the inner 14 and outer 12 cannulas are provided with control flanges 18 arranged to cooperate with one another to facilitate control of the device 10 by a physician and, more particularly, to enable the physician to move the two cannulas relative to one another. In the illustrated embodiment, these control flanges 18 are provided in the form of curved surfaces extending from the sides of the inner 14 and outer 12 cannulas. Advantageously the control flanges 18 of the outer cannula 12 curve generally toward the distal end of the device 10 and are positioned on opposite sides of the outer cannula 12. The control flange 18 of the inner cannula 14 is positioned at the most proximal point of the inner cannula 14 and curves away from the remainder of the device 10. Thus, it will be seen that the respective control flanges 18 of the inner 14 and outer 12 cannulas actually extend away from one another. This arrangement facilitates grasping of the outer cannula 12 control flanges 18 by a physician's fingers, typically the index and middle fingers. The control flanges 18 of the inner cannula 14 are ideally placed for engagement by the physician's thumb. In this manner, the hand of the physician is properly positioned to retract the outer cannula 12 relative to the inner cannula 14 by pulling/pushing the index and middle fingers and the thumb toward one another.

The inner cannula 14 preferably includes a distal opening 20 through its surface at a desired location. This opening 20 allows for communication between the exterior and interior of the inner cannula 14 and will generally be located adjacent the distal end of the inner cannula 14. More particularly, this opening 20 allows for surgical instruments inserted into the inner cannula 14 to interact with the environment exterior to the inner cannula 14 and within the treatment area. The location, shape, and size of the opening 20 will be variable depending upon the nature of the procedure for which the surgical device 10 will be used. As a non-limiting example, the opening 20 referenced above takes the form of an open slot in the upper wall of the inner cannula 14.

In the illustrated embodiment, the inner cannula 14 is provided with a proximal orifice 24 to allow the introduction of a first surgical instrument into the inner cannula 14. The proximal orifice is aligned with the long axis of the inner cannula 14 and is positioned at approximately the midpoint of the inner cannula's control flange 18.

As can be seen most clearly in FIG. 1, the inner cannula 14 is also provided with a horizontally elongated slot 26 extending nearly the entire length of the underside of the inner cannula 14. This elongated slot 26 allows communication between the interior of the inner cannula 14 and the outer cannula 12. As previously described, the outer cannula 12 is provided with an open top 28 that further facilitates this communication between the cannulas 12, 14. More particular, the combination of the elongated slot 26 of the inner cannula 14 and the open top 28 of the outer cannula 12 allows instruments that are introduced into the outer cannula 12 to be extended into the interior space of the inner cannula 14 and into the treatment area through the distal opening 20 of the inner cannula 14.

The outer cannula 12 is arranged to expand and contract relative to the inner cannula 14. When contracted, the interior walls of the outer cannula 12 lies substantially against the exterior surfaces of the inner cannula 14. This position minimizes the total exterior thickness of the device 10, thereby easing insertion and extraction of the device 10 through an incision. In the expanded position, the total, combined, interior space of the device 10—the space encompassed by the interior walls of both the inner 14 and outer 12 cannulas is maximized to allow for the introduction of multiple instruments into that interior space. This allows both a scope and another surgical instrument to be used by the physician at the same time. In the illustrated embodiment, this movement is accomplished by means of a tab and slot combination incorporated into the respective walls of the inner 14 and outer 14 cannulas.

More particularly, each side of the inner cannula 14 is provided with a pair of tabs 32 that extend from the sides of the inner cannula 14. Each pair of tabs 32 is in alignment along or parallel to the long axis of the inner cannula 14. In the illustrated embodiment, the tabs 32 are roughly cylindrical in shape. Each side of the outer cannula 12 is provided with a pair of slots 34 that are arranged to engage the tabs 32 of the inner cannula 14. The slots 34 are preferably angled relative to the long axis of the outer cannula 12. This angled orientation results in the outer cannula 12 moving away from the inner cannula 14 as the outer cannula 12 is retracted. When the outer cannula 12 is advanced relative to the inner cannula 14, the orientation of the slots 34 results in the outer cannula 12 moving closer to the inner cannula 14. Thus, this movement gives rise to the expansion and contraction of the device 10 as referenced above.

Advantageously, the angled slots 32 are provided with a distal portion 36 that is aligned with the long axis of the outer cannula 12 rather than continuing the angled orientation. Further, this portion 36 of the angled slots 34 may also be provided with a slightly constricted region 38. The constricted region 38 has a width no more than equal to, and preferably slightly less than, the diameter of the tabs 32. Thus, the constricted region 38 serves as a locking detent to hold the outer cannula 12 in the expanded position away from the inner cannula 14 until the user applies sufficient advancing force to move the tabs 32 past the constricted region 38 and into the angled portion of the slot 34 to allow contraction of the device 10.

In a preferred embodiment, the distal end of the inner cannula 12 is substantially aligned with the distal end of the outer cannula 14 when advanced into the contracted position. This position is particularly suitable for initial insertion of the device 10 into an incision and to the desired treatment area within the patient. The expanded position of the outer cannula 12, in which the outer cannula 12 has been moved away from the lower surface of the inner cannula 14, allows for introduction of multiple instruments through the combined cannulas. For example, a scope may be introduced through the entry guide 30 of the outer cannula 12 while a second surgical instrument is introduced through the proximal orifice 24 of the inner cannula 14. Furthermore, the retraction of the outer cannula 12 can serve to gently separate tissues within the treatment area to provide additional room for the physician to maneuver the device 10 during the procedure and greater visibility within the treatment area.

While one embodiment of the expansion/contraction mechanism for the outer cannula 12 has been described herein, other mechanisms are possible and are considered to be within the scope of the present invention. For example, a hinged connection between the outer cannula 12 and inner cannula 14 may be used in which the hinges are located along one side of the device 10 and the outer cannula 12 is rotated relative to the inner cannula 14 to increase the available space within the device 10. In this embodiment, the outer cannula 12 does not retract and advance relative to the length of the inner cannula 14.

In addition, a dial and rack and pinion version of the expansion/contraction mechanism is also possible. In this embodiment, the rack may be attached to an exterior lateral wall of the inner cannula 14 while the pinion is connected to a lateral wall of the outer cannula. A dial or other device to allow a user to turn the pinion is connected thereto. The pinion may be oriented at an angle relative to the axes of the inner and outer cannulas to produce an expansion and contraction function similarly to the angled slot and tab described above.

Further, an additional embodiment is possible in which the expansion/contraction mechanism utilizes the structure of a speculum with two spaced handles connected with respective operating ends by a pivot point. Squeezing the handles together results in the operating ends, in this case the inner and outer cannulas, being separated from one another.

The dimensions of the inner 14 and outer 12 cannulas and the interior spaces thereof may be varied based on the specific procedure for which the device 10 will be used and the type and size of the instruments with which it will be used.

In another embodiment, the device 10 is provided with a light source that produces a targeting light in the treatment area. The light source may take a variety of forms. For example, a fiber optic light may be inserted into either the proximal orifice 24 of the inner cannula 14 or the entry guide 30 of the outer cannula 12 and extended through the device 10 such that it will project light out of the distal end of the device 10.

In another embodiment, the light source is incorporated into the walls of either the inner 14 or outer 12 cannula in the form of a passage extending longitudinally through such wall from the proximal end to the distal end of the device 10. At the distal end of the device, the light source passage terminates in a translucent port. In a particularly advantageous embodiment, this port is tinted to produce a more distinct light, resulting in a “bullseye” being highlighted on a surface of the treatment area. A surgical light source, for example, and fiber optic lamp, is connected with the proximal end of the light source passage for illumination. Alternately, a fiber optic lamp is incorporated directly into the light source passage.

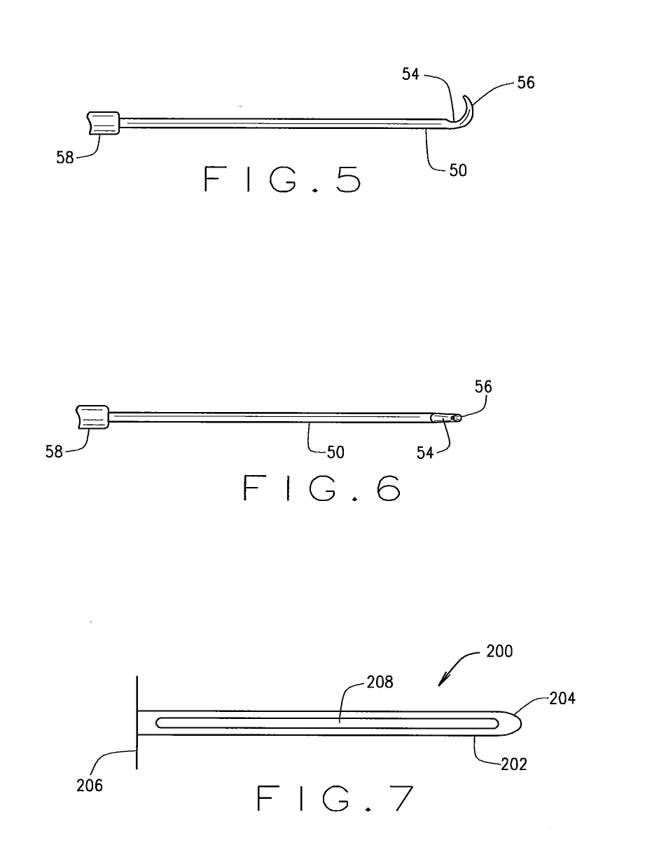

As shown in FIGS. 5 and 6, the illustrated device 10 further includes cartridge 50 that is arranged to move within the inner and outer cannulas. The use of a cartridge provides the benefit, among others, of allowing the introduction of multiple instruments, for example as shown in the figures, a scope and a surgical tool. However, the device 10 may be utilized without the cartridge 50. In such embodiments, instruments would be introduced directly into the inner and outer cannulas, and the opening(s) of the cannulated member are sized to accommodate those instruments appropriately. In other embodiments, the cartridge 50 is utilized without the inner and outer cannulas.

The proximal end of the cartridge 50 may be provided with flanges or extensions to enhance control of the cartridge 50. An open viewing portal 54 is provided in the surface of the cartridge 50 near its distal end. The cartridge 50 is arranged to accommodate a surgical scope, e.g. an endoscope, arthroscope, or laproscope, within it. The scope is inserted into the open interior of the cartridge 50 toward the distal end. The open viewing portal 54 accommodates the lens of the surgical scope and provides for a view out of the cartridge 50.

When the cartridge 50 and inner cannula are properly aligned with one another, the open viewing portal 54 of the cartridge 50 and the open slot 20 of the inner cannula cooperate to provide a clear view out of the device 10 to the treatment area within the patient. The relative arrangement of the inner and outer cannulas and the open slot allow for unobstructed viewing when the cartridge 50 is in either of the cannulas.

In a preferred embodiment, the cartridge 50 also has sleeve 58 for engagement with the base of a scope to secure the cartridge 50 and scope to one another during the procedure. The sleeve 58 will generally be a cylindrical piece of flexible material into which the base of the scope can be inserted. A rubber or similar material having resistance to slippage is preferred.

The cartridge 50 may also be provided with one or more surgical tools that are selected for a particular procedure and are known within the field of endoscopic, arthroscopic, or laparoscopic surgery. In the case of the illustrated embodiment, a cartridge 50 suitable for endoscopic plantar fasciotomy (“EPF”), or similar cutting applications, is shown. This form of the cartridge 50 is provided with a cutting blade 56 at the distal end. This particular version has a hooked blade specific to EPF and designed to cut bands of the plantar fascia. This particular version may also have applicability in carpal tunnel release and gastroc resection procedures, among others.

It should be noted that this is a non-limiting example of the possible variations of the cartridge 50. Variations of the cartridge 50 can be provided with different probes, curettes, grabbers, biters, biopsy tools, cauterization tips, punches, needles, and drills, and all of these variations are considered to be within the scope of the present invention. The possible attachments to the cartridge include devices that serve as electrocautery probes for removal of unwanted or harmful tissue, coagulation of bleeding tissue, and sealing blood vessels to help reduce or stop bleeding. The electrocautery probe function can be incorporated into the tips of the previous listed instruments, e.g., hook, grabber, needle, drill, etc.

Advantageously, the device 10 may be manufactured as a disposable set that is ready for immediate one-time use in a sterile or other setting.

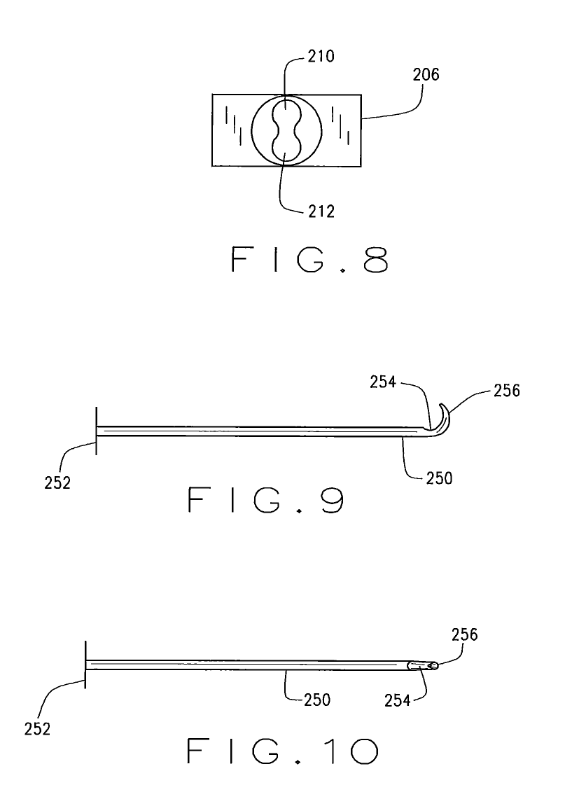

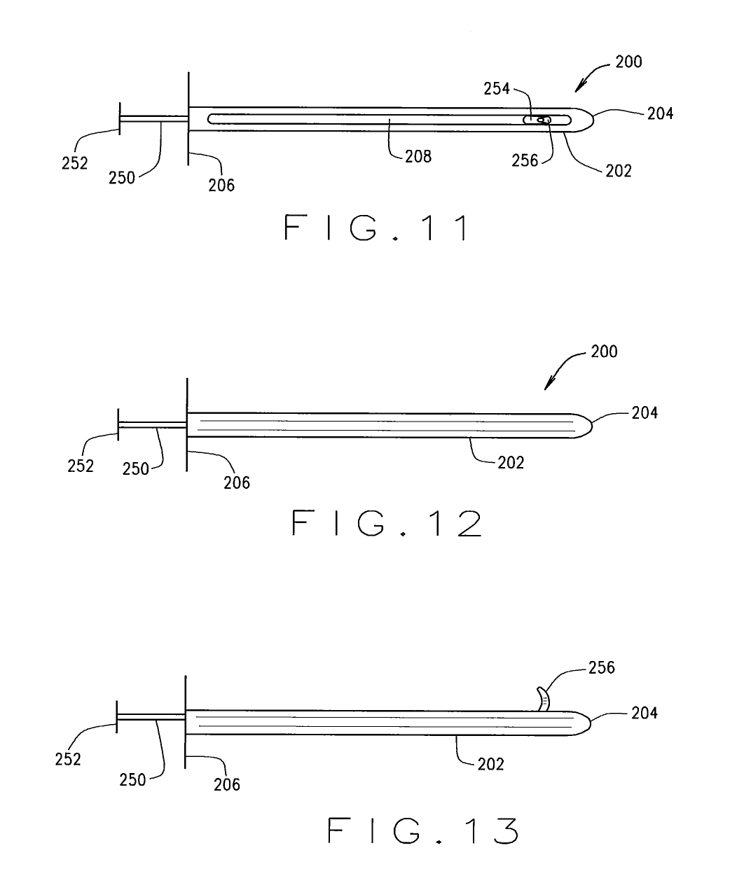

FIGS. 7-13 illustrate another embodiment of a multi-function cannulated surgical device 200. In this embodiment, the inner and outer cannulas are fixed relative to one another. In pertinent part, the device 200 includes an elongated cannulated member 202. In this embodiment, the inner cannula 210 is represented by the upper portion of the cannulated member 202, while the outer cannula 212 is represented by the lower portion of the member 202. In this embodiment, the inner and outer cannulas are fixed relative to one another and may be formed from a single piece. The distal end of the cannulated member 202 is provided with a rounded tip 204 that eases insertion of the device through an incision. In alternate embodiments, the cannulated member 202 may be provided with a spatula tip. The proximal end of the cannulated member 202 is provided with flanges 206 or similar extensions that enhance control of the device by a physician.

The cannulated member 202 preferably includes an opening through its surface at a desired location. This opening allows for communication between the exterior and interior of the cannulated member 202. More particularly, this opening allows for tools inserted into the cannulated member 202 to interact with the environment exterior to the outer cannula and within the treatment area. The location, shape, and size of the opening will be variable depending upon the nature of the procedure for which the surgical device 200 will be used.

As a non-limiting example, an embodiment suitable for endoscopic plantar fasciotomy is illustrated in FIGS. 12-18. In this particular embodiment, the opening referenced above takes the form of an open slot 208 through the surface of the cannulated member 202 and extending along its length. This open slot 208 communicates with the interior of the cannulated member 202, which is formed with inner 210 and outer 212 cannulas. The inner 210 and outer 212 cannulas are in communication with one another. When viewed from the proximal end of the cannulated member 202, the inner 210 and outer 212 cannulas have a roughly hourglass shaped or “double barreled” cross section. The cannulas 210, 212 are open to one another at the narrow portion of the hourglass shape. While the cannulas 210, 212 are shown in a vertical arrangement in the illustrated embodiment, they may also be arranged in a horizontal arrangement. However, in each embodiment, one of the cannulas will be in direct communication with the open slot 208.

While the cannulas 210, 212 are shown in FIGS. 12-18 as communicating with one another, various embodiments will incorporate a cannulated member in which the cannulas are isolated from one another. In such embodiments, the cannulated member may possess two openings—one of which communicates with the inner cannula while the second communicates with the outer cannula—in order to allow instruments introduced into each cannula to interact with the environment exterior to the cannulated member. Variations of the cannulated member in which more than two cannulas, for example, three or four, may be utilized to accommodate additional instruments and/or to allow for additional flexibility in the amount of the treatment area surrounding the cannulated member with which the surgeon may interact with various instruments simultaneously, while maintaining the need for only a single incision to access the treatment area.

The dimensions of the cannulated member 202, the inner 210 and outer 212 cannulas, and the open slot 208, or other opening(s) in the cannulated member, may be varied based on the specific procedure for which it will be used and the type and size of the instruments with which it will be used. As a non-limiting example, the cannulated member 202 of the illustrated embodiment is approximately 8 mm in diameter and approximately 10 cm in length. Each of the inner 210 and outer 212 cannulas is approximately 3 mm in diameter.

As shown in FIGS. 14 and 15, the illustrated device 200 further includes cartridge 250 that is arranged to move within the cannulated member 202. The use of a cartridge provides the benefit, among others, of allowing the introduction of multiple instruments, for example as shown in the figures, a scope and a surgical tool. However, the cannulated member 202 may be utilized without the cartridge 250. In such embodiments, instruments would be introduced directly into the cannulas of the cannulated member, and the opening(s) of the cannulated member are sized to accommodate those instruments appropriately. In other embodiments, the cartridge 250 is utilized without the cannulated member 202.

Returning to the illustrated embodiment, the cartridge 250 will have a diameter slightly less than the diameter of the inner 210 and outer 212 cannulas of the cannulated member 202. Advantageously, however, the diameter of the cartridge 250 will be slightly larger than the width of the narrow passage joining the inner 210 and outer 212 cannulas. This sizing of the cartridge 250 relative to the cannulas 210, 212 results in the cartridge 250 being retained within one of the cannulas at any given time and requiring application of a small degree of force to the cartridge 250 to move it from one cannula to the other. This provides positive positioning of the cartridge 250 within the cannulated member 202 and prevents unintended migration of the cartridge 250 between the cannulas 210, 212.

Much like the cannulated member 202, the cartridge 250 has a roughly extended tube-like shape with an open interior. The proximal end of the cartridge 250 is also provided with flanges 252 or extensions to enhance control of the cartridge 250. An open viewing portal 254 is provided in the surface of the cartridge 250 near its distal end. The cartridge 250 is arranged to accommodate a surgical scope, e.g. an endoscope, arthroscope, or laproscope, within it. The scope is inserted into the open interior of the cartridge 250 toward the distal end. The open viewing portal 254 accommodates the lens of the surgical scope and provides for a view out of the cartridge 250.

As illustrated in FIG. 16, when the cartridge 250 and cannulated member 202 are properly aligned with one another, the open viewing portal 254 of the cartridge 250 and the open slot 208 of the cannulated member 202 cooperate to provide a clear view out of the device 200 to the treatment area within the patient. In the illustrated embodiment, the relative arrangement of the inner 210 and outer 212 cannulas and the open slot 208 allow for unobstructed viewing when the cartridge 250 is in either of the cannulas 210, 212.

The cartridge 250 may also be provided with one or more surgical tools that are selected for a particular procedure and are known within the field of endoscopic and arthroscopic surgery. In the case of the illustrated embodiment, a cartridge 250 suitable for endoscopic plantar fasciotomy (“EPF”), or similar cutting applications, is shown. This form of the cartridge 250 is provided with a cutting blade 256 at the distal end. This particular version has a hooked blade specific to EPF and designed to cut bands of the plantar fascia. This particular version may also have applicability in carpal tunnel release and gastroc resection procedures, among others.

It should be noted that this is a non-limiting example of the possible variations of the cartridge 250. Variations of the cartridge 250 can be provided with different probes, curettes, grabbers, biters, biopsy tools, cauterization tips, punches, needles, and drills, and all of these variations are considered to be within the scope of the present invention.

In the illustrated embodiment, the blade 256 is arranged at the distal tip of the inner cannula 250. The blade 256 extends upward and in the same direction as the open viewing portal 254 faces. Advantageously, this allows a physician to utilize the blade (or other tool) without interference of the surgical scope.

The hourglass shape of the interior of the cannulated member 202 allows for significant control and enhanced safety during procedures. In particular, positioning of the cartridge 250 in the lower canal, which is more distant from the open slot 208, effectively “disengages” the cutting blade 256 in the illustrated embodiment (and other tools in variations of the cartridge 250) by retracting the blade 256 into the cannulated member 202. This prevents contact of the blade 256 with patient tissue and any resulting unintended cutting of tissue. At the same time, the surgical scope retains a clear view of the treatment area. Because additional force must be applied in order to move the inner cannula from the bottom to the top canal, accidental contact of the blade 256 or other tool with patient tissue is minimized. When the physician has confirmed that the device 200 is in the proper position for the required procedure, the cartridge 250 may be moved into the inner cannula 210 for cutting.

The length of the open slot 208 allows the cartridge 250, and, consequently, the surgical scope and blade 256 or other tool, to be positioned anywhere along the length of the cannulated member 202 without need to reposition the cannulated member 202.

As a non-limiting example of use of the illustrated device 200, the steps involved in using the device in an EPF procedure are provided. The physician first makes a 1 cm incision on the medial aspect of the patient's foot, at or just proximal to the high point of the arch of the foot. Blunt dissection lateral to the middle band of the plantar fascia using the cannulated member 202 then occurs. Next, the cartridge 250 is fitted with an endoscope, e.g. a 2.7 mm/70°/4″ scope. The cartridge 250 with the inserted scope is introduce into the outer cannula 212 to allow for viewing of the plantar fascia without cutting. When the cartridge 250 is in the proper position, it is raised into the inner cannula 210 to allow the physician to see and cut the fascia as required. When cutting is complete, the cartridge 250 is dropped back down to the outer cannula 212 and retracted from the cannulated member 202. The cannulated member 202 is then removed through the incision.

Advantageously, the device 200 may be manufactured as a disposable set that is ready for immediate one-time use in a sterile or other setting.

In general, embodiments of the present invention are particularly well-suited for a variety of applications, including for example endoscopic procedures such as fascial release—plantar, carpal, ulnar, etc. —muscle release—gastroc/soleous, etc.; arthroscopic procedures on large joints (hip, knee, shoulder), medium joints—ankle, subtalar, etc., and small joints—carpal, meta carpal, metatarsal, phalangeal, etc.; laparoscopic procedures such as gastroenterological (GI) procedures, urological, general surgery, and obstetrics and gynecological, e.g. hysteroscopy; minimally invasive surgery (MIS) procedures such as spine, orthopedic, plastics and cosmetic procedures, otolaryngological procedures, and cardiac procedures.

The preferred embodiments of the invention have been described above to explain the principles of the invention and its practical application to thereby enable others skilled in the art to utilize the invention in the best mode known to the inventors. However, as various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by the above-described exemplary embodiment, but should be defined only in accordance with the following claims appended hereto and their equivalents.

What is claimed is:

1. A multi-function, cannulated, surgical device, comprising:
an inner cannula, said inner cannula defining an opening therein to allow communication between an interior space of said inner cannula and an environment exterior to said surgical device;
an outer cannula connected with said inner cannula; and
wherein said inner and outer cannulas define a shared slot therebetween to allow communication between said interior space of said inner cannula and an interior space of said outer cannula.

2. The multi-function, cannulated, surgical device as set forth in claim 1, wherein said opening defined by said inner cannula defines an exterior slot.

3. The multi-function, cannulated surgical device as set forth in claim 1, wherein said inner and outer cannulas are fixed relative to one another.

4. The multi-function, cannulated, surgical device as set forth in claim 1, wherein said inner and outer cannula are movable relative to one another.

5. The multi-function, cannulated, surgical device as set forth in claim 4, wherein said outer cannula is connected with said inner cannula by means allowing controlled movement of said outer cannula toward and away from said inner cannula, while maintaining a connection between said inner and outer cannula, to increase a combined interior space within said inner and outer cannulas.

6. The multi-function, cannulated, surgical device as set forth in claim 5, wherein said means for allowing movement further allows retraction and advancement of said outer cannula relative to said inner cannula.

7. The multi-function, cannulated, surgical device as set forth in claim 5, wherein said means for allowing movement comprises at least one tab located on an exterior, lateral surface of said inner cannula and an angled slot in a lateral wall of said outer cannula and wherein said tab engages said angled slot.

8. The multi-function, cannulated, surgical device as set forth in claim 5, wherein said means for allowing movement comprises at least one tab located on an interior, lateral surface of said outer cannula and an angled slot in a lateral wall of said inner cannula and wherein said tab engages said angled slot.

9. The multi-function, cannulated, surgical device as set forth in claim 5, wherein said means for allowing movement comprises a hinged connection between said outer cannula and said inner cannula.

10. The multi-function, cannulated, surgical device as set forth in claim 1, further comprising a cartridge that is slidingly engageable with one of said inner and said outer cannulas, a surgical tool being connected with a distal end of said cartridge.

11. The multi-function, cannulated, surgical device as set forth in claim 10, wherein said surgical tool is selected from the group consisting of a cutting blade, a probe, a curette, a grabber, a biter, a biopsy tool, a cauterization tip, a punch, a needle, and a drill.

12. The multi-function, cannulated, surgical device as set forth in claim 10, further comprising an electrocautery probe incorporated into said surgical tool.

13. The multi-function, cannulated, surgical device as set forth in claim 1, further comprising at least a first control flange connected with said cannulated member.

14. The multi-function, cannulated, surgical device as set forth in claim 1, further comprising a light source incorporated into a wall of one of said cannulated member.

15. The multi-function, cannulated, surgical device as set forth in claim 14, wherein said light source communicates with a tinted port at a distal end of said cannulated member.

16. The multi-function, cannulated, surgical device as set forth in claim 14, wherein said light source comprises a fiber optic lamp.

17. The multi-function, cannulated, surgical device as set forth in claim 10, further comprising a light source connected with said cartridge.

18. The multi-function, cannulated, surgical device as set forth in claim 17, wherein said light source communicates with a tinted port at a distal end of said cartridge.

19. The multi-function, cannulated, surgical device as set forth in claim 17, wherein said light source comprises a fiber optic lamp.

20. The multi-function, cannulated surgical device as set forth in claim 10, wherein said cartridge further comprises a sleeve for connected a scope with said cartridge.
